(19) United States
(12) Patent Application Publication
(10) Pub. No.: US 2005/0131031 A1
(43) Pub. Date: Jun. 16, 2005

(54) Method of producing a nicotine composition

(76) Inventor: Marshall Anlauf Thompson, Camarillo, CA (US)

Correspondence Address: KELLY BAUERSFELD LOWRY & KELLEY, LLP, 6320 CANOGA AVENUE, SUITE 1650, WOODLAND HILLS, CA, 91367, US

(21) Appl. No.: 11/045,953

(22) Filed: Jan. 27, 2005

Related U.S. Application Data
(63) Continuation-in-part of application No. 10/216,023, filed on Aug. 9, 2002.
Application No. 10/216,023 is a continuation-in-part of application No. 10/015,101, filed on Dec. 10, 2001, now abandoned.

(51) Int. Cl. A24F 47/00; A61K 31/44

(52) U.S. Cl. 514/343; 426/422

(57) ABSTRACT

A method of producing a consumable nicotine composition includes blending a measured quantity of nicotine into a solution. A nicotine concentration is achieved in the solution of between 0.0001% and 0.1% and the solution apportioned so that each portion contains a precise quantity of nicotine appropriate for consumption in a single use by a single person. Each portion is introduced into a respective single-serving beverage. Another method of producing a consumable nicotine composition also includes blending a measured quantity of nicotine into a solution and achieving a nicotine concentration in the solution of between 0.0001% and 0.1%. The solution is likewise apportioned so that each portion contains a precise quantity of nicotine appropriate for consumption in a single use by a single person. Each portion is introduced into a respective beverage and each beverage packaged into a separate single-use dispenser.

### RELATED APPLICATION

This is a continuation-in-part of U.S. Patent Application Ser. No. 10/216,023, filed Aug. 9, 2002, which is a continuation-in-part of U.S. Patent Application Ser. No. 10/015,101, filed Dec. 10, 2001.

### BACKGROUND OF THE INVENTION

The present invention relates generally to nicotine beverages that serve as cigarette substitutes for individuals attempting to quit smoking. More particularly, the present invention relates to a method for producing a consumable composition having a precise, single-serving quantity of nicotine using conventional beverage production equipment.

When the cigarette-rolling machine was patented in 1881, it enabled a relatively minor product of the time, the cigarette, to become an extremely popular product. It increased cigarette production by 5000 percent and cut the price of an individual cigarette in half. However, the increase in the cigarette's popularity also brought with it a host of health-related problems.

Today, many people attempting to quit smoking have turned to substitute products containing nicotine. Doctors have long recommended that individuals attempting to quit smoking should consume large amounts of fluids. Fluid consumption in itself is necessary for individual health. Additionally, persons who stop smoking often misinterpret cravings for a cigarette as hunger, which frequently results in weight gain. Drinking fluids helps suppress the cravings for a cigarette and helps curb weight gain. Also, drinking fluids incorporates the same hand-to-mouth motion associated with smoking, which may be psychologically helpful to persons attempting to quit smoking.

In light of the foregoing, an especially effective cigarette alternative is the nicotine beverage. In addition to the advantages listed above, it also has a built-in protection against nicotine abuse, because the nicotine concentration in a beverage can be adjusted so that an individual will reach his or her fluid intake limit before consuming a dangerous amount of nicotine.

As noted above, the cigarette-rolling machine enabled a huge increase in cigarette production as well as a substantial decrease in production cost, thereby allowing the cigarette to become an extremely popular product. The object of the present invention is to have a similar effect on the nicotine beverage market through a method which allows for increased beverage production as well as decreased production costs. This will result in nicotine beverages becoming more available, less expensive, and more popular with consumers.

It is well known that nicotine is a potent substance. Very small amounts of nicotine can produce noticeable effects in adult humans. In fact, the amount of nicotine appropriate for human consumption in a single use, i.e. the amount required to effectively serve as a single cigarette substitute, is too small to be accurately measured in a conventional beverage production setting. The equipment is not sufficiently precise and any error in measurement could mean lethal results for consumers of the beverage.

It is possible to measure nicotine into single-serving amounts by utilizing precision equipment. However, such equipment is expensive and normally unavailable in conventional beverage production settings. In order to be an attractive substitute for cigarettes, a nicotine beverage must be relatively inexpensive. The use of precision measuring equipment will result in higher-priced nicotine beverages, which will be less attractive to individuals attempting to quit smoking.

Accordingly, there is a need for a method of inexpensively producing a composition containing a precise amount of nicotine appropriate for a single use by a single individual. Such a method should not require the use of high-precision measuring equipment, but should instead utilize equipment already found in conventional beverage production settings. Additionally, to increase marketability, such a method should be capable of yielding a variety of compositions, including liquids, solids, tablets, pills, and powders. The present invention fulfills these needs and provides other related advantages.

### SUMMARY OF THE INVENTION

The present invention resides in a method for producing a composition containing a precise amount of nicotine appropriate for a single use by a single individual. The method utilizes equipment normally found in conventional beverage production settings, and functions by blending nicotine into one or more successive intermediate solutions then dividing the resulting liquid nicotine solution until yielding a final composition appropriate for a single-serving beverage. This method of dividing a large intermediate liquid nicotine composition eliminates the need for expensive, high-precision measurement equipment. As a result, nicotine beverages will be less expensive to produce and will have the potential to reach a larger market.

A method of producing a consumable nicotine composition embodying the present invention includes blending a measured quantity of nicotine into a solution. A nicotine concentration is achieved in the solution of between 0.0001% and 0.1%. The solution is apportioned so that each portion contains a precise quantity of nicotine appropriate for consumption in a single use by a single person. Each portion is introduced into a respective single-serving beverage.

The blending step includes the step of measuring a quantity of nicotine comprising at least 50 mg of nicotine.

The nicotine comprises a component of a liquid extracted from various natural sources including, without limitation, tobacco leaf, finely cut tobacco, or powdered tobacco. The nicotine (C10H14N2) may also be obtained from a non-natural source.

A primary component of the beverage may comprise various liquids/fluids that include, without limitation, water (carbonated or non-carbonated) or a natural juice.

The solution may be in various forms including, without limitation, a liquid or a non-liquid.

The portions are introduced into the beverages prior to sale or distribution.

The beverage includes an alcoholic constituent comprising at most 50% alcohol. The beverage is placed into a container.

Another method of producing a consumable nicotine composition that embodies the present invention includes blending a measured quantity of nicotine into a solution. A nicotine concentration is achieved in the solution of between 0.0001% and 0.1% and the solution is apportioned so that each portion contains a precise quantity of nicotine appropriate for consumption in a single use by a single person. Each portion is introduced into a respective beverage and each beverage packaged into a separate single-use dispenser.

The single-use dispensers are sold or distributed to be introduced into beverages by consumers. The single-use dispenser may come in various forms including, without limitation, an eyedropper, a porous or semi-porous pouch, similar to a tea bag, a packet, a capped tube, or a dissolvable pill or tablet.

A nicotine concentration of between 0.0001% and 0.1% by volume is attained in the beverage after adding the contents of the single-use dispenser thereto.

Additional features and advantages of the present invention will become apparent from the following more detailed description.

### DETAILED DESCRIPTION OF THE INVENTION

The present invention is a method for producing a composition containing a precise amount of nicotine appropriate for a single use by a single individual. The nicotine composition may be introduced into a single-serving beverage container in a production setting, or it may be packaged in a single-use dispenser and sold to consumers. Regardless of how the composition is made available, the method of producing the composition remains the same and is the subject of the present invention.

The method involves blending a known quantity of nicotine of 50 mg or greater into at least one intermediate solution until the desired concentration of nicotine is attained. The nicotine concentration must be blended or otherwise diluted as 50 mg or more of nicotine is considered an amount that is hazardous and/or lethal for human consumption. With each successive intermediate solution, the nicotine concentration is progressively reduced. The solution is divided into smaller volumes that maintain the same nicotine concentration as the larger volume. For example, it is known that a cigarette contains roughly 1 to 4 mg of nicotine and that about 1 mg of nicotine is inhaled from a single cigarette. Therefore, a nicotine beverage ought to contain a roughly equivalent amount by delivering about 1 mg of nicotine per serving. Since 1 mg of nicotine is too small to be accurately measured in a conventional beverage production setting, the method of the present invention may be employed, for example, as follows: First, a known quantity of nicotine (e.g., 50 mg) is mixed/blended into a measured quantity of water, or other solvent, to produce an intermediate solution. This known quantity is easily measurable in a beverage production setting. The nicotine concentration in this intermediate solution may not be within the desired concentration ratio of nicotine to water or some other solvent. In that case, additional intermediate solutions may be needed to attain the desired nicotine concentration of between 0.0001% and 0.1% in the final solution.

Once the desired concentration is attained, the final solution is then divided into a number of small equal portions by volume while maintaining the desired nicotine concentration. Each portion contains a precise quantity of nicotine such that the nicotine content of the final portioned solution is appropriate for a single use by a singe person.

The portions containing the final nicotine composition may be introduced into a beverage that is then placed into single beverage containers (e.g., cans, bottles, jugs or the like) in a production setting prior to sale or distribution, or the final nicotine composition may be packaged in single-use dispensers (e.g., packets, tubes, cans, bottles or the like) to be mixed into beverages later. In the latter case, the final nicotine composition may even be evaporated to yield the single-serving quantity of nicotine contained therein, which may then be mixed with a water-soluble powder filler and sealed in a packet. Such a packet would contain an appropriate amount of nicotine for a single serving and could be opened and its contents mixed into any beverage of choice by the consumer or person serving the beverage.

The nicotine may be a component of a liquid extracted from various natural sources including, without limitation, tobacco leaf, finely cut tobacco (e.g., moist snuff), or powdered tobacco (e.g., dry snuff). The nicotine (C10H14N2) may also be obtained from a non-natural source.

A primary component of the beverage may comprise various liquids/fluids that include, without limitation, water (carbonated or non-carbonated) or a natural juice. The beverage may include an alcoholic constituent of up to at most 50% alcohol.

The solution may be in various forms including, without limitation, a liquid or a non-liquid.

It should be noted that while water is specifically mentioned in describing the blending/dilution and apportioning process, the method of the present invention may employ a wide range of substances as a solvent. It should also be noted that the method of the present invention may be manipulated to yield a final nicotine composition that is a liquid, a solid, a tablet, a pill, a powder, or any other desired form.

The single-use dispenser may come in various forms including, without limitation, an eyedropper (with or without a bottle containing the nicotine solution), a porous or semi-porous pouch, similar to a tea bag, a packet, a capped tube, or a dissolvable pill or tablet.

Although embodiments of the present invention have been described in detail for purposes of illustration, various modifications may be made without departing from the scope and spirit of the invention. Accordingly, the invention is not to be limited, except as by the appended claims.

1. A method of producing a consumable nicotine composition, comprising:
blending a measured quantity of nicotine into a solution;
achieving a nicotine concentration in the solution of between 0.0001% and 0.1%;
apportioning the solution so that each portion contains a precise quantity of nicotine appropriate for consumption in a single use by a single person; and
introducing each portion into a respective single-serving beverage.

2. The method of claim 1, wherein the blending step includes the step of measuring a quantity of nicotine comprising at least 50 mg of nicotine.

3. The method of claim 1, wherein the nicotine comprises a component of a liquid extracted from tobacco leaf.

4. The method of claim 1, wherein the nicotine comprises a component of a liquid extracted from finely cut tobacco.

5. The method of claim 1, wherein the nicotine comprises a component of a liquid extracted from powdered tobacco.

6. The method of claim 1, including the step of obtaining the nicotine (C10H14N2) from a non-natural source.

7. The method of claim 1, wherein a primary component of the beverage comprises water.

8. The method of claim 1, wherein a primary component of the beverage comprises carbonated water.

9. The method of claim 1, wherein a primary component of the beverage comprises a natural juice.

10. The method of claim 1, wherein the solution comprises a liquid.

11. The method of claim 1, wherein the solution comprises a non-liquid.

12. The method of claim 1, including the step of introducing the portions into the beverages prior to sale or distribution.

13. The method of claim 1, wherein the beverage includes an alcoholic constituent comprising at most 50% alcohol.

14. The method of claim 1, including the step of placing the beverage into a container.

15. A method of producing a consumable nicotine composition, comprising:
blending a measured quantity of nicotine into a solution;
achieving a nicotine concentration in the solution of between 0.0001% and 0.1%;
apportioning the solution so that each portion contains a precise quantity of nicotine appropriate for consumption in a single use by a single person;
introducing each portion into a respective beverage; and
packaging each beverage into a separate single-use dispenser.

16. The method of claim 15, wherein the single-use dispensers are sold or distributed to be introduced into beverages by consumers.

17. The method of claim 15, wherein the single-use dispenser comprises an eyedropper.

18. The method of claim 15, wherein the single-use dispenser comprises a porous or semi-porous pouch, similar to a tea bag.

19. The method of claim 15, wherein the single-use dispenser comprises a packet.

20. The method of claim 15, wherein the single-use dispenser comprises a capped tube.

21. The method of claim 15, wherein the single-use dispenser comprises a dissolvable pill or tablet.

22. The method of claim 15, wherein a nicotine concentration of between 0.0001% and 0.1% by volume is attained in the beverage after adding the contents of the single-use dispenser thereto.
